(19) United States
(12) Patent Application Publication
(10) Pub. No.: US 2023/0022685 A1
(43) Pub. Date: Jan. 26, 2023

(54) PRESSURE DECONTAMINATION SYSTEM PROVIDING FAST CLEANING AGAINST CBRN THREATS SUITABLE FOR USE IN MILITARY VEHICLES AND INFRASTRUCTURES

(71) Applicant: Nero Endüstri Savunma Sanayi Anonim Sirketi, Sincan, Ankara (TR)

(72) Inventor: Alican ÖKÇÜN, Sincan, Ankara (TR)

(21) Appl. No.: 17/785,474

(22) PCT Filed: Jun. 15, 2021
(86) PCT No.: PCT/TR2021/050600

§ 371 Date: Jun. 15, 2022

(30) Foreign Application Priority Data
Jun. 17, 2020 (TR) 2020/09387

(51) Int. Cl. A61L 2/18; A61L 2/04; B08B 9/08

(52) U.S. Cl. CPC A61L 2/18; A61L 2/04; B08B 9/0865; B08B 9/0804; A61L 2202/15; B08B 2209/08

(57) ABSTRACT

A system which can be manually/automatically activated to ensure instant cleaning of decontamination liquid and gas placed into cylindrical pressure tubes against CBRN threats, wherein the system includes: a decontamination liquid/gas used for cleaning chemical, biologic, radiological and nuclear threats and minimizing effects thereof, a decontamination tube used for discharging decontamination liquid/gas placed therein, a decontamination pyrotechnic igniter-based on pyrotechnic used for ignition of the decontamination tube, a control unit providing manual or automatic operation of the system, and at the same time communication thereof with the hardware units in the system, and a control card located on the control unit, having printed circuit boards with embedded software developed for enabling the control.

### TECHNICAL FIELD

The invention relates to improving a cleaning (decontamination) system which comprises redundant cleaning tube (double-shot system) for providing system security and which can be manually/automatically activated to ensure instant cleaning of decontamination liquid and gas placed into the cylindrical pressure tubes against CBRN (chemical, biologic, radiological and nuclear) threats, and use thereof in military vehicles and indoor area.

### BACKGROUND OF THE INVENTION

Today, there is known decontamination systems. In these systems, in case of emergency, the related person/environment is interfered by the personnel with protective clothing, thus the exposure time of the current personnel to the chemical or biological agent is much longer. There are permanent damages because of CWAs (Chemical Warfare Agents) and TIA (Toxic Industrial Agents), Biological agents, Radiological and Nuclear materials especially specified in the scope of CBRN.

In the present applications, there is no system based on fast reaction and automation for the solution of the system and the agents in the employed systems cannot be used quickly for the person exposed to the threat.

In the present systems, CBRN threats are not possible to be eliminated in a very short time in an automatic/semi-automatic mode through communicating automatically with a system and sensor devices.

U.S. Pat. No. 9,827,601B2 is revealed through technical research and summarized as follows; “A method for efficiently decontaminating surfaces is provided comprising applying an indicator to a surface, wherein the indicator provides an observable or machine readable response when a contamination is present on the surface, wherein the response is located relative to a location of the contamination; and decontaminating the location of the contamination, and optionally rechecking the location of the initial contamination post decontamination to ensure that the surface is free of contamination.”

Patent Document No. EP3427761A2 is revealed through technical research and summarized as follows; “A method for management of complex emergencies and contamination sites in order to recover a delimited area affected by fire and industrial or pathogenic, chemical and/or biological and/or nuclear and/or radiological toxic agents, which area is delimited by at least one boundary wall, the method comprising the following steps: a) perforating said boundary wall by generating at least one hole; b) extinguishing the fire by dispensing a jet of a fire-extinguishing liquid through the hole; c) decontaminating/detoxifying/disinfecting the area by dispensing a jet of an operation fluid through the hole. Said operation liquid being supplemented with a decontamination/detoxification product.”

As can be seen, the systems of foregoing patent applications are related to some decontamination methods, however there is no mention of an embodiment that can provide a solution to the disadvantages mentioned above.

As a result, due to abovementioned drawbacks and the insufficiency of present solutions in the art, it is necessary to make an improvement in the related technical field.

### OBJECT OF THE INVENTION

The invention aims to provide a structure having different technical features that are novel in this field, different from the embodiments used in the known state of art.

The main object of the invention is to set forth a system which can be manually/automatically activated to ensure instant cleaning of decontamination liquid and gas placed into the cylindrical pressure tubes against CBRN (chemical, biologic, radiological and nuclear) threats.

An object of the invention to provide discharging decontamination liquid in less than 1 second with pressure tubes and to minimize the level of harm to personnel affected by CBRN (Chemical Biological, Radiological and Nuclear) threats and to passivate CBRN contamination on the related vehicle/equipment before affecting with fast activation.

Another object of the invention is, to enable the use of the redundant tube where secondary decontamination process is required by providing the redundant tube in addition to each and every tube placed into the vehicle/area while the cleaning operation is realized manually or automatically. Thanks to this feature, the system is provided with a possibly required second cleaning operation subsequent to first cleaning operation thus a higher protection is provided.

Another object of the invention is to provide semi-automatic or fully automatic decontamination of critical personnel who must fulfill their duty and who does not have protective equipment during duty such as filter and etc., where needed through fast activation with automation and pyrotechnic blaster system of pressure tubes, thus to minimize the harm that develop instantly.

Therefore, the invention aims to carry out abovementioned objects through a system which can be manually/automatically activated to ensure instant cleaning of decontamination liquid and gas placed into the cylindrical pressure tubes against CBRN threats, wherein the system comprises;
Decontamination liquid/gas used for cleaning chemical, biologic, radiological and nuclear threats and minimizing effects thereof,
Decontamination tube used for discharging decontamination liquid/gas placed therein,
Decontamination pyrotechnic igniter based on pyrotechnic used for ignition of decontamination tube,
Control unit providing manual or automatic operation of the system, and at the same time communication thereof with the hardware units in the system,
Control card located on the control unit (4) and consists of printed circuit boards comprising embedded software developed for enabling the control.

The structural and characteristic features and all the advantages of the invention will be understood more clearly by means of the figures and the detailed description with reference to these figures given below and therefore, the evaluation should be made by taking these figures and the detailed description into consideration.

### FIGURES FOR A BETTER UNDERSTANDING OF THE INVENTION

FIG. 1, a representative view of the decontamination tube according to the invention.

FIG. 2, a representative view of the control unit.

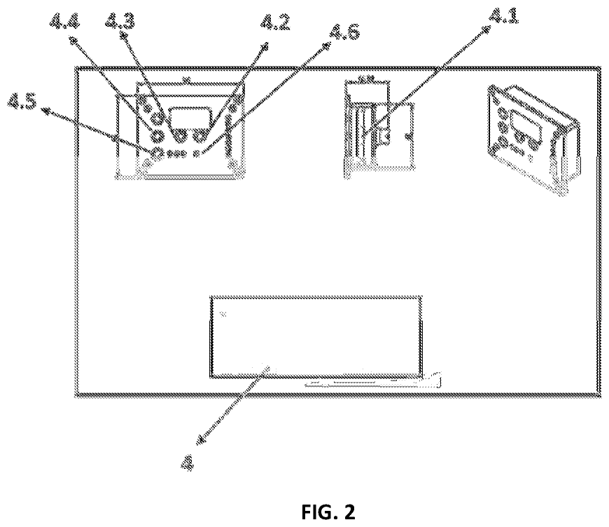

FIG. 3, a view of the exemplary layout plan of the decontamination tubes according to the invention.

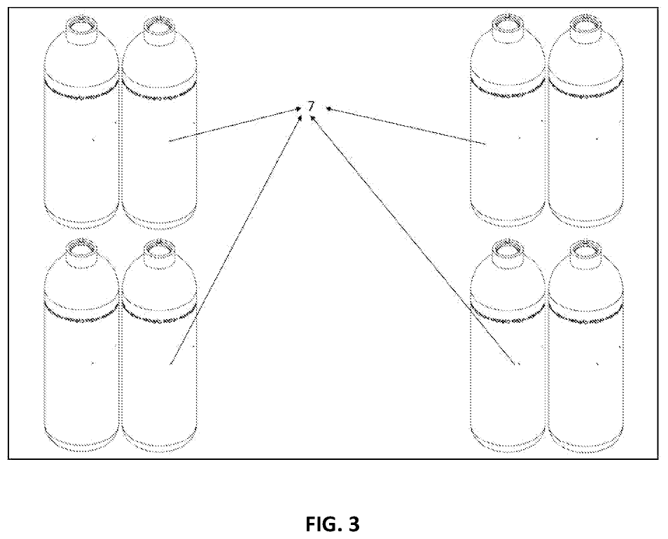

The drawings are not necessarily drawn to scale and details which are not necessary for the understanding of the present invention may be omitted. In addition, elements that are substantially identical or have substantially identical functions are denoted by the same reference signs.

### LIST OF THE REFERENCE NUMBERS

1. Decontamination tube
2. Decontamination pyrotechnic igniter
3. Decontamination liquid/gas
4. Control unit
4.1 Control card
4.2 Automatic mode indicator
4.3 Manuel mode indicator
4.4 Pressure state indicator
4.5 Redundant tube indicator
4.6 Power indicator/button
5. Decontamination tube manometer
6. Decontamination tube pressure membrane
7. Decontamination redundant tube

### DETAILED DESCRIPTION OF THE INVENTION

In this detailed description, the preferred embodiments of the invention are merely described for a better understanding of the subject matter and without any limiting effect.

The invention relates to improving a cleaning (decontamination) system which comprises redundant cleaning tube (double-shot system) for providing system security and which can be manually/automatically activated to ensure instant cleaning of decontamination liquid and gas placed into the cylindrical pressure tubes against CBRN (chemical, biologic, radiological and nuclear) threats, and use thereof in military vehicles and indoor area.

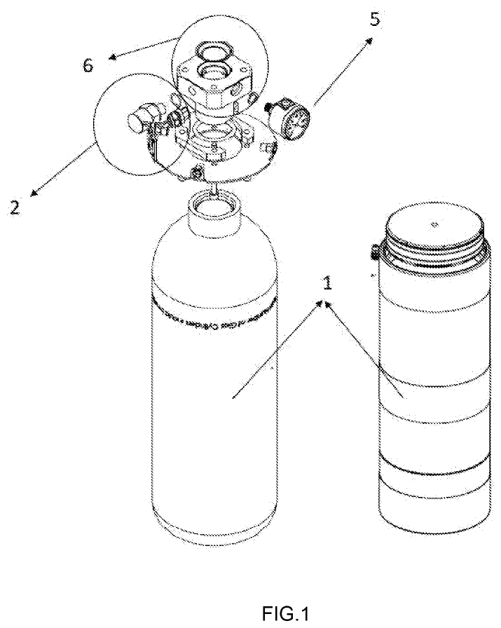

The decontamination tube (1) is a military type of tube that provides discharging of the decontamination liquid within 500 milliseconds in total and that can operate between −40+85 degrees due to its material strength. Since each of the decontamination tubes (1) is placed with respect to their impact area, a blind spot on the vehicle/area is prevented. As can be seen from FIG. 1, it can be in different sizes whether in large and small volumes.

Decontamination pyrotechnic igniter (2) is a pyrotechnic based igniter blaster enabling the decontamination tube (1) to be ignited. When the electrical signal received from the control unit (4) is delivered to the pressure membrane on the tube by the pyrotechnic method; decontamination pyrotechnic igniter (2) acts as a structure which is chemically activated, and which ejects the metal particle thereon in order to perforate the membrane. Therefore, membrane is perforated, and decontamination liquid/gas placed into the tube is discharged.

The discharge unit is a unit which comprises decontamination pyrotechnic igniter (2) and other mechanical equipment that discharges the agent within the tube. These are units which receive electrical signals.

The decontamination liquid/gas (3) is a special mixture of liquids/gases used for cleaning Chemical, Biological, Radiological and Nuclear threats and minimizing impact thereof.

The decontamination tube pressure indicator (5) is an indicator used for pressure tracking of the decontamination tube (1).

The decontamination tube pressure membrane (6) is a metal based membrane material to be opened with a decontamination pyrotechnic igniter (2) for the retention at high pressure in the tube and fast discharge of the liquid/gas (3) placed into the decontamination tube (1).

The control unit (4) is a unit which provides manual and automatic operation of the system and at the same time communication thereof with the other hardware units. The control card (4.1) located on the control unit is a system which consists of printed circuit boards comprising embedded software developed for enabling the control.

The control unit (4) is able to operate in integration with CBRN agent detection system which is located on the vehicle and used as redundant and with the electronic components and processors thereon, and when necessary, in the case where the automatic mode is open, it is able to operate automatically with the signal from the CBRN sensors and detection systems. Also, by means of a switch on the control unit (4), a selection is made possible whether manually or automatically and the user may activate the system manually as desired.

There are the automatic mode indicator (4.2) and the manual mode indicator (4.3) indicating the operating mode, the pressure status indicator (4.4) enabling tracking the pressure status, the redundant tube indicator (4.5) enabling indication of whether the redundant contamination tube (7) is used or not and a power indicator/button (4.6) indicating the operation status of the system and/or starting and ending its operation on the control unit (4).

The redundant decontamination tube (Double shot system) (7) are tubes providing secondary cleaning in order to ensure reliable decontamination process.

The decontamination system according to the invention have an operation principle as follows:

The system, which is under CBRN threats, discharges its pressure solution to the related person or area when necessary and cleans the contamination, manually or automatically.

In this context, it is aimed that delivering decontamination liquid/gas (3) placed into the decontamination tube (1) to the area to be cleaned and instantly cleaning the contamination through opening the decontamination tube pressure membrane (6) located on the tube by means of the pyrotechnic igniter system (2) with the signal from the control unit (4) sent manually or automatically. When the pressure of the decontamination tube (1) is lower or higher than the targeted value, it is ensured that information is gathered before a critical situation by transferring the related pressure value on the pressure sensor on the tube to the control unit (4) by means of the decontamination tube pressure indicator (5) tracking the instantaneous error status of the system.

If the information gathered from redundant tube configuration located on the system and the other hardware systems placed on the vehicle shows high level CBRN contamination, it is provided minimizing the possibility of secondary harm by exploding the redundant decontamination tube (7).

The system according to the invention, the operation steps realized as follows;
The production of the decontamination tube (1) (It is important to produce the decontamination tube (1) with compatible material for the use in the targeted military conditions).
Deploying and positioning the decontamination pyrotechnic igniter (2) system on the outlet of the decontamination tube (1) suitably, (With regard to the most efficient discharge of extinguishing liquid/gas and performance of the cleaning process it is important to deploy decontamination pyrotechnic igniter suitably).
Selection of the decontamination liquid with regard according to the risk of danger, (Cleaning performance should be maximized by selecting the right decontamination liquid/gas (3)).
Filling the decontamination liquid/gas (3) into the pressure decontamination tube (1), (It holds a critical importance to ensure that filling the decontamination liquid/gas (3) into the tube with the correct pressure for wrong activations and reaching the sufficient speed).
Pressurizing the decontamination liquid with the suitable pressure for the discharge of 500 milliseconds in total, (It holds a critical importance to ensure that filling the decontamination liquid/gas (3) into the tube with the correct pressure for wrong activations and reaching the sufficient speed).
Positioning the mechanical devices that provides stable placement of the of the decontamination fluid in the vehicle/area (It is important to ensure the mechanically stable placement of the system in order not to experience problems during the cleaning process).
Designing and producing the control unit (4) and control card (4.1) so as to enable automatic or manual operation, (It holds a critical importance that the control unit and card (4, 4.1) is produced in a sufficient configuration.)
Integration of the redundant decontamination tubes (7) for the decontamination operation (It holds a critical importance that positioning of the redundant tubes correctly for the requirement of secondary cleaning).

1. A system which is manually/automatically activated to ensure instant cleaning of decontamination liquid and gas placed into cylindrical pressure tubes against CBRN threats, the system comprising:
decontamination liquid/gas used for cleaning chemical, biologic, radiological and nuclear threats and minimizing effects thereof;
a decontamination tube used for discharging decontamination liquid/gas placed therein;
a decontamination pyrotechnic igniter based on pyrotechnic used for ignition of the decontamination tube;
a control unit providing manual or automatic operation of the system, and at the same time communication thereof with the hardware units in the system; and
a control card located on the control unit and comprising of printed circuit boards comprising embedded software developed for enabling the control.

2. The system according to claim 1, comprising a redundant decontamination tube (Double shot system) providing secondary cleaning in order to reliably provide decontamination operation.

3. The system according to claim 1, comprising a decontamination tube pressure indicator used for pressure tracking of the decontamination tube.

4. The system according to claim 1, comprising a decontamination tube pressure membrane which opens with the decontamination pyrotechnic igniter for the retention at high pressure in the tube and fast discharge of the liquid/gas placed into the decontamination tube.

5. The system according to claim 1, comprising an automatic mode indicator and a manual mode indicator indicating the operating mode which is located on the control unit.

6. The system according to claim 1, comprising pressure status indicator providing tracking the pressure status from the control unit.

7. The system according to claim 1, comprising a redundant tube indicator providing indication of whether the redundant contamination tube is used or not.

8. The system according to claim 1, comprising a power indicator/button indicating the operation status of the system and/or starting and ending its operation.

9. A method providing manual/automatic activation to ensure instant cleaning of decontamination liquid and gas placed into cylindrical pressure tubes against CBRN threats, characterized by comprising the operation steps as follows:
producing a decontamination tube;
deploying and positioning a decontamination pyrotechnic igniter system on an outlet of the decontamination tube;
selection of the decontamination liquid with regard according to the risk of danger;
filling the decontamination liquid/gas into the pressure decontamination tube;
pressurizing the decontamination liquid with the suitable pressure for the discharge of 500 milliseconds in total;
positioning the mechanical devices that provides stable placement of the automatic decontamination fluid in the vehicle/area;
designing and producing a control unit and control card so as to enable automatic or manual operation; and
integration of the redundant decontamination tubes for the decontamination operation.
